(19) United States
(12) Patent Application Publication
(10) Pub. No.: US 2009/0012524 A1
(43) Pub. Date: Jan. 8, 2009

(54) A CUTTING TOOL FOR USE IN ORTHOPAEDIC SURGERY

(75) Inventor: Liam Dower, Huddersfield (GB)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: DEPUY INTERNATIONAL LIMITED, Leeds (GB)

(21) Appl. No.: 10/575,396

(22) PCT Filed: Sep. 15, 2004
(86) PCT No.: PCT/GB2004/003917

§ 371 Date: Sep. 23, 2008

(30) Foreign Application Priority Data
Oct. 8, 2003 (GB) 0323478.8

(51) Int. Cl. A61B 17/00

(52) U.S. Cl. 606/80; 606/79

(57) ABSTRACT

A cutting tool for preparing a cavity in a bone for receiving a component of an orthopaedic joint prosthesis. The shape of the tool is based on a shell having a rotationally symmetrical outer surface. The tool has at least one portion cut out from it, the cut out portion extending from the peripheral edge of the shell toward the pole of the shell, such that the tool has no more than one plane of symmetry passing through the axis of rotation. The external surface of the shell presents at least two outwardly directed cutting teeth, arranged such that the net translational force on the tool in the plane which is perpendicular to the axis of rotation, resulting from the accumulated resistance of the teeth when rotated against a rotationally symmetrical cavity in which the tool is a snug fit, is approximately balanced.

This invention relates to a cutting tool for preparing a cavity in a bone for receiving a component of an orthopaedic joint prosthesis.

A rotationally symmetrical cavity in a bone can be prepared to receive a component of an orthopaedic joint prosthesis using a cutting tool which is rotationally symmetrical. The cutting tool will usually be an approximately hemispherical shell which is hollow. It will include a formation in its interior so that it can be engaged by an instrument by which rotational movement can be imparted to the shell. The formation can include for example a bar which extends across the shell. The axis of rotation of the shell will usually be coincident with the polar axis of the shell. The peripheral edge of the shell will usually define a plane which is perpendicular to the polar axis. The angle subtended at the centre of the sphere, of which the surface of the shell forms a part, between the peripheral edge at two diametrically opposite points will generally be close to 180°, for example at least about 170°. The cutting tool will have cutting teeth on its external spherical surface so that it cuts the bone to form the cavity by rotation when in contact with the bone. The cutting teeth can be in the form of elongate slits, or generally round perforations. Such cutting tools are known, for example as disclosed in U.S. Pat. No. 5,203,653 and U.S. Pat. No. 5,709,688. Preferably, the arrangement of the cutting teeth on the surface of the tool is such that the axis around which torque, arising from the accumulated resistance to rotation of the tool when rotated against bone tissue, is applied to the tool is approximately coincident with the polar axis of the tool.

Cutting tools of this kind are used after preparatory steps which include forming an incision and removal or displacement of any obstructing tissue. A tool is mounted on a suitable drive instrument which can be used to impart rotational movement to the tool and inserted through the incision to engage the relevant surface of the bone.

It can be desirable to minimise the size of an incision through which access is gained to a patient's bone to perform surgery on it, especially in order to reduce the period taken for the patient to recover from the surgery. The size of a cutting tool, when mounted on the drive instrument, can determine the minimum size of the incision in a procedure. It can therefore be desirable to reduce the size of a shell cutting tool, while still ensuring that the tool can cut bone to form a cavity of desired shape and size. Current cutting tools exist which have ‘cutout’ portions so that the shell is able to fit through smaller incisions as its effective width is reduced compared with a full hemispherical shell.

However, it has been found that when cutting tools which have cutout portions arranged such that the cutting tool only has one plane of symmetry passing through the axis of rotation, are rotated against the bone to prepare a cavity in the bone, the surgeon experiences a ‘wobbling’ or ‘vibration’ effect, wherein the cutting tool vibrates within the cavity being formed.

The vibration effect experienced by the surgeon causes discomfort to the surgeon using the tool. Further, the vibration of the tool in the cavity can cause a cavity to be formed in the bone which is larger than the volume defined by the hemispherical shell from which the tool is formed, and also which is irregular in shape. This is highly undesirable as a cavity needs to have precise dimensions so that a prosthetic component can be snugly received within the cavity.

The present invention provides a hemispherical shell cutting tool, in which the shell has at least one portion cut out from it such that the peripheral edge of the shell is intact at two diametrically opposite points, and in which the teeth are arranged on the external spherical surface such that the net translational force on the tool resulting from the accumulated resistance of the teeth when rotated against the bone tissue is approximately balanced.

Accordingly, in one aspect, the invention provides a cutting tool for preparing a cavity in a bone for receiving a component of an orthopaedic joint prosthesis, in which the shape of the tool is based on a shell having a rotationally symmetrical outer surface, the tool having at least one portion cut out from it, the cut out portion extending from the peripheral edge of the shell toward the pole of the shell, such that the tool has no more than one plane of symmetry passing through the axis of rotation, in which the external surface presents at least two outwardly directed cutting teeth, arranged such that the net translational force on the tool in the plane which is perpendicular to the axis of rotation, resulting from the accumulated resistance of the teeth when rotated against a rotationally symmetrical cavity in which the tool is a snug fit, is approximately balanced.

The tool of the invention has the advantage that its effective width is reduced compared with a full hemispherical shell by virtue of the cutout portions. However, the size of the recess which can be formed in a bone using the cutting tool is determined by the shape of the tool in the regions in which the peripheral edge is intact: this can be the same as in a conventional hemispherical shell in which there are no cutout portions. Accordingly, it is possible by virtue of the tool of the invention for the tool to be inserted through an incision that is smaller than is necessary with a conventional full hemispherical shell, and to obtain a recess which has the same shape as that which is obtained with the conventional shell.

The cutout portions that are features of the tool of the invention mean that there can be fewer cutting teeth by which to form the cavity in the bone. However, it has been found according to the present invention that adequate cutting is possible with the tool of the invention, notwithstanding the loss of some of its external surface on which cutting teeth can be arranged.

It is an advantage of the present invention that, by arranging the teeth on the surface such that the net translational force on the tool, provided by the teeth resisting motion of the tool as they cut into the bone, is approximately balanced, the tool can be rotated during the cutting operation without the generation of unacceptable transverse movement, which can manifest itself as vibration during rotation of the tool.

Preferably the full width of the shell is preserved at two diametrically opposed points. This allows the shell to include a bar extending across the interior of the shell, from one side to the opposite other side, by which the shell can be engaged by an instrument for manipulation. The bar can be provided at the open face of the shell. The bar can be recessed within the shell, at a point between the open face and the interior surface at the pole. The use of a bar to provide a connection between a cutting tool shell and a rotational drive instrument is well established and features which are known in this context for forming such a connection can be used in the present invention.

The cutting tool of the invention can have at least one cut out portion, at least two cut out portions, at least three cut out portions, at least four cut out portions, or more. The number of cut out portions will be selected according to the size of the incision through which the tool is to be inserted to engage the patient's bone, and to the cutting area which is to be retained on the external surface of the shell.

Preferably, the shape, size and location of the cut out portions are such that the centre of mass is on the axis of rotation of the tool.

Preferably, the peripheral edge of the shell is intact around at least about 30% of the length of the circumference of the shell at the edge, more preferably at least about 35%, especially at least about 40%. Preferably, the peripheral edge of the shell is intact around not more than about 80% of the length of the circumference of the shell at the edge, more preferably not more than about 70%, especially not more than about 60%.

Preferably, at least one of the cut out portions extends over at least about 15% of the length of the circumference of the shell at the peripheral edge, more preferably at least about 20%, for example at least about 25%. This can enable appreciable reductions in the transverse dimension of the tool to be achieved.

Preferably, the edges of the cut out portions where the cut out portions meet the intact peripheral edge of the shell extend radially towards the pole of the shell. However, the cut out portions can have different shapes. For example, each cut out portion can be defined by a chord which extends directly from one edge of that cut out portion, where it meets the intact peripheral edge of the shell, to its opposite edge. Other shapes of cut out portion are also envisaged. For example, a curved edge can extend from one side of a cut out portion, where it meets the intact peripheral edge of the shell, to its opposite side.

Preferably, the size of one of the cut out portions differs from the size of another of the cut out portions. It will often be the case that, in order to achieve balance of the tool with the centre of mass on the polar axis, the size of two of the cut out portions will be approximately the same and different from the size of the or each other cut out portions.

Preferably, the size of one of the portions of the shell in which the peripheral edge is intact differs from the size of another of the said portions. It will often be the case that, in order to achieve balance of the tool with the centre of mass on the polar axis, the size of two of the intact portions will be approximately the same and different from the size of the or each other intact portions.

The angle subtended at the centre of the sphere, of which the surface of the shell forms a part, between the peripheral edge at the said diametrically opposite points will generally be less than 180° so that the tool does not generate a cavity whose diameter at the open face is less than the maximum diameter of the tool. Preferably, the said angle is at least about 150°, preferably at least about 170°.

Preferably, the shell is intact in a circular region around the pole. This has the advantage of optimising the area of the external surface of the shell in the region in which maximum axial force will have to be withstood. This also has the advantage of optimising the surface that is available for providing cutting teeth and also optimising the strength of the tool so that to enable it to withstand applied axial forces. Preferably, the ratio of the distance from the edge of the circular region to the pole to the ratio of the distance of the peripheral edge of the shell to the pole (both distances being measured along the spherical surface of the shell) is not more than about 0.5, more preferably not more than about 0.4, for example about 0.3. Preferably, the ratio of the distance from the edge of the circular region to the pole to the ratio of the distance of the peripheral edge of the shell to the pole (both distances being measured along the spherical surface of the shell) is not less than about 0.1, more preferably not less than about 0.2.

The tool of the invention will generally have at least two cutter teeth which protrude outwardly from the spherical surface of the shell. A cutter tooth can be formed by cutting the material of the shell and deforming the material outwardly. The exposed edge of the cut material should be sharp in order to be able to cut the bone tissue, either as a result of the cutting step or as a result of a subsequent sharpening step. A cutter tooth can be in the form of an elongate slit. Preferably, the tool of the invention has cutter teeth in the form of generally round perforations, especially a plurality of the said teeth arranged over the external surface of the shell. Each such cutter tooth is preferably formed by cutting the material of the shell part way around the edge of a generally round opening, and deforming the material of the shell by bending it at that part of the edge of the opening where the material is not cut. The transverse dimension of such an opening (which will be a diameter when the opening is circular) is preferably at least about 1.5 mm, more preferably at least about 2.0 mm, especially at least about 2.5 mm.

It is an advantage to use a plurality of circular openings for the cutter teeth because it allows the distribution of cutter teeth over the surface of the tool to be varied. For example, it can be preferred to have a greater concentration of the cutter teeth close to the pole of the tool, and compared with close to the peripheral edge of the tool.

Preferably, the cutting teeth are distributed approximately evenly over the surface of the shell so that the number of teeth per unit area of the external surface thereof is approximately constant over the entire surface. It will be appreciated however that this can only be approximated because of the relative sizes of the teeth and the surface of the shell.

Preferably, all cutting teeth have identical shape and dimensions. Alternatively, the cutting teeth may not all have identical shape and dimensions.

Preferably, the cutting teeth are arranged such that the net translational force on the tool in the plane which is perpendicular to the axis of rotation, resulting from the accumulated resistance of the teeth when rotated against a rotationally symmetrical cavity in which the tool is a snug fit, is approximately balanced.

Preferably, the cutting teeth are arranged into sets of cutting teeth. Preferably, there is at least one set of teeth. More preferably, there are two sets of teeth. Especially preferably, there are three sets of teeth, or more.

Preferably, any of the sets of cutting teeth may consist of two, three, four or more cutting teeth. The sets of teeth can comprise different numbers of teeth.

Preferably, the teeth within each set of teeth are arranged on the contacting surface such that the net translational force on the tool in the plane which is perpendicular to the axis of rotation, resulting from the accumulated resistance of the teeth of each set when rotated against a rotationally symmetrical cavity in which the tool is a snug fit, is approximately balanced. However, the arrangement of the teeth on the intact part of the shell in the circular region around the pole of the tool need not be arranged such that net translational force on the tool is approximately balanced.

Preferably, the teeth within any of the sets of teeth are the same distance from the axis of rotation of the tool, and the length of an are taken between any pair of adjacent teeth within any of the sets is equal for each pair of adjacent teeth within that set.

Preferably, the teeth are arranged on the contacting surface in the form of a spiral. More preferably, the teeth are arranged in the form of two spirals. Especially preferably, the teeth are arranged in the form of three spirals. More especially preferably, the teeth are arranged in the form of four spirals or more.

Preferably, the teeth are arranged in the form of an interrupted spiral. More preferably, the teeth are arranged in the form of two interrupted spirals. Especially preferably, the teeth are arranged in the form of interrupted three spirals. More especially preferably, the teeth are arranged in the form of four interrupted spirals or more.

It is an advantage for the teeth to be arranged in the form of a spiral or an interrupted spiral, as it has been found that when the teeth are arranged as such, the cutting tool is ‘pulled’ into the bone as the tool cuts the bone.

Preferably, the teeth are arranged so that there is an overlap between the teeth as you move from the equator to the pole of the shell of the cutting tool. It is an advantage for the teeth to be arranged as such, as when the tool is rotated about its axis there will be at least one tooth cutting over the hemispherical surface being produced without the need to wiggle the cutting tool.

The tool of the invention can be made from materials which are suitable for surgical cutting tools. Particularly preferred materials include certain stainless steels. The tool can be formed by casting. When the tool includes a bar which extends across the interior of the shell, this can be formed integrally with the shell in a casting operation, or separately and then fastened to the shell, for example by welding. Cutter teeth can be formed on the shell in a casting step, or separate from the step of forming the shell, for example in a subsequent operation involving for example cutting or machining or both.

Embodiments of the invention will now be described by way of example with reference to the accompanying drawings, in which:

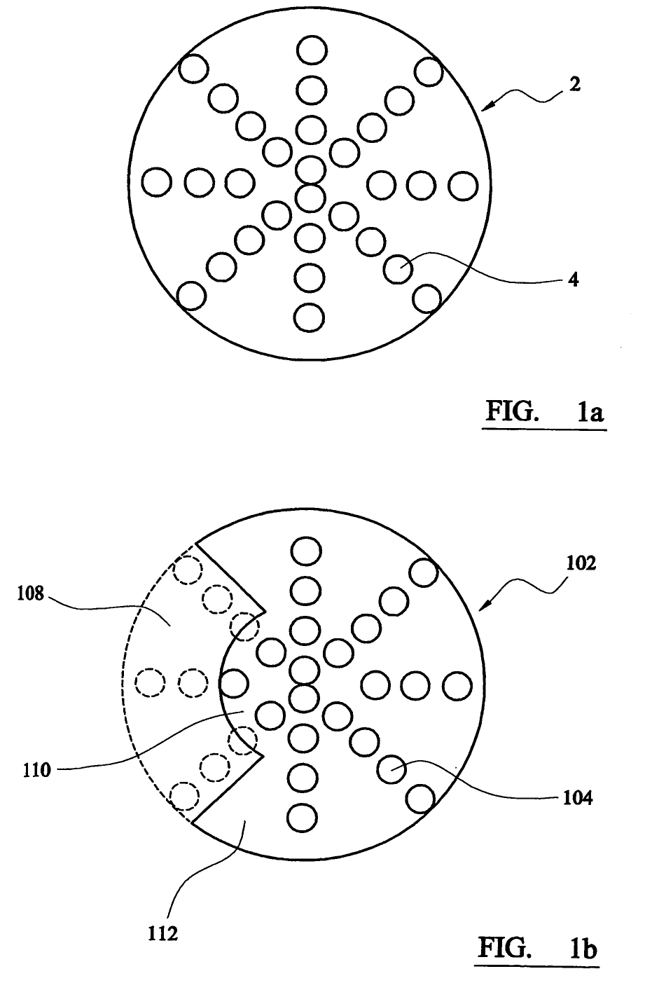

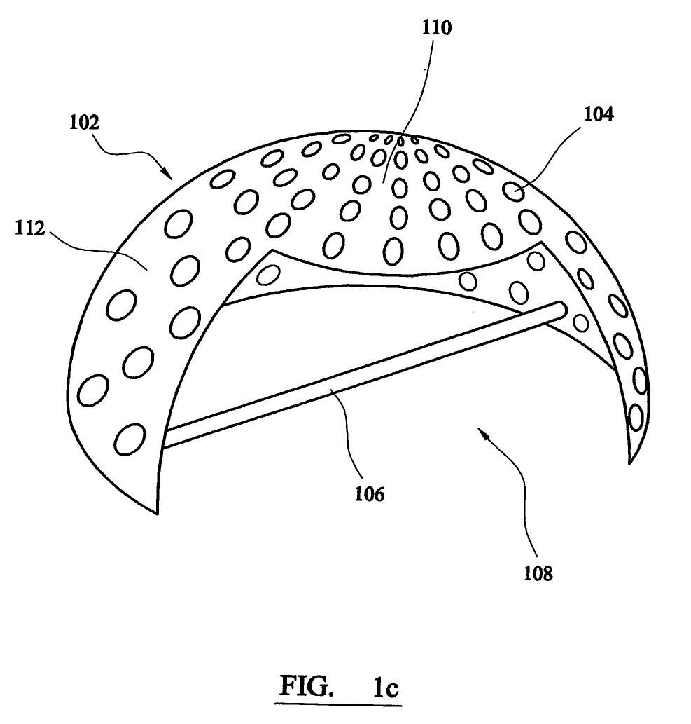

FIG. 1a shows a schematic plan view of a first existing cutting tool,

FIG. 1b shows a schematic plan view of a second existing cutting tool having a cut out portion,

FIG. 1c shows an isometric view of the cutting tool of FIG. 1b,

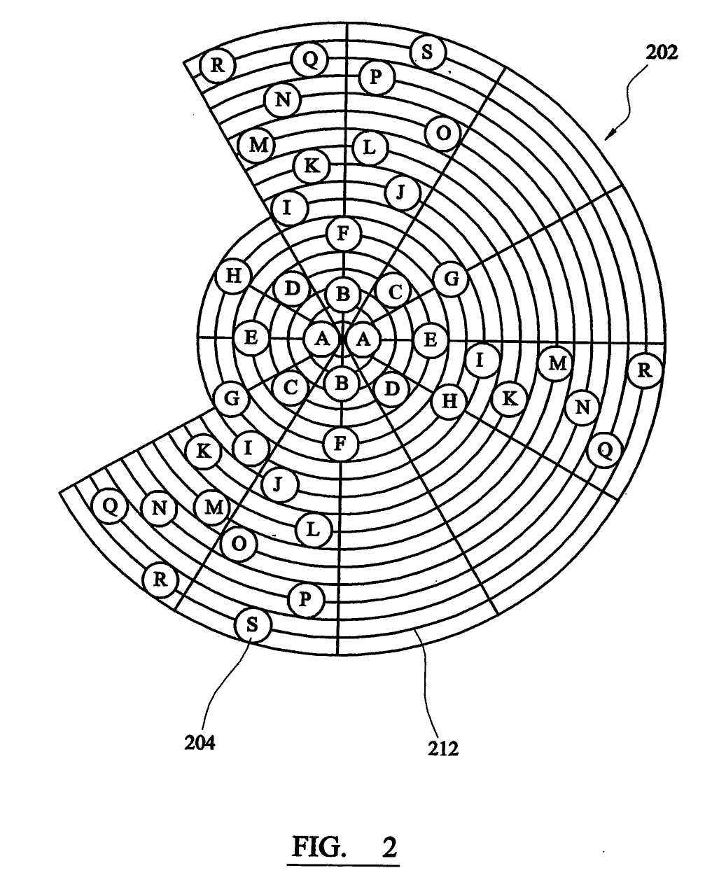

FIG. 2 shows a schematic plan view of a cutting tool according to one embodiment of the invention,

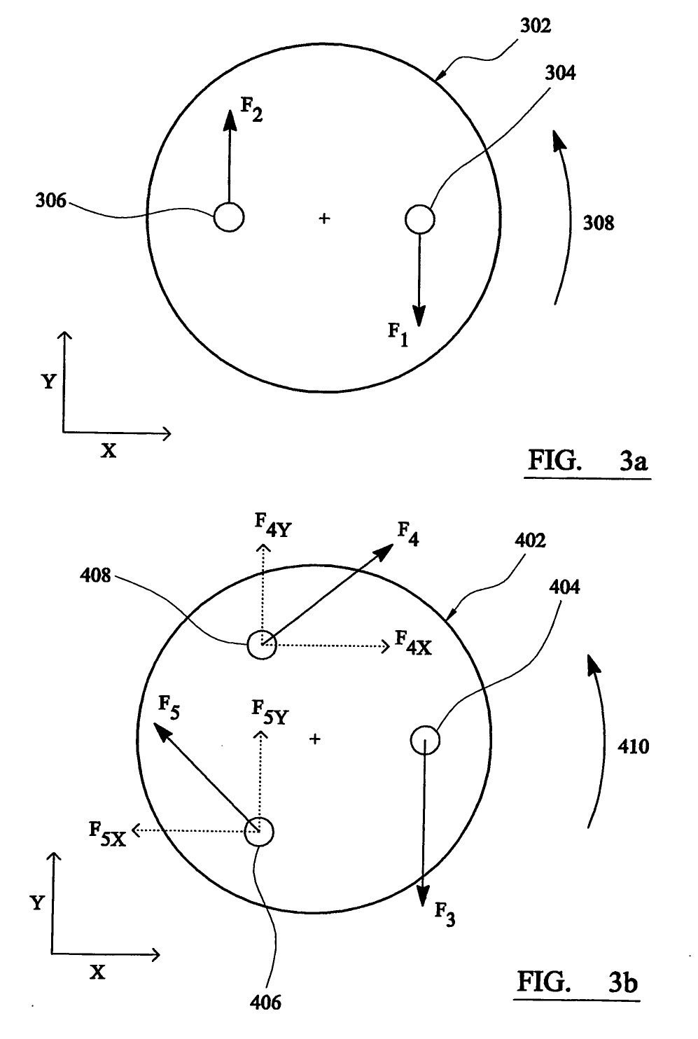

FIGS. 3a and 3b illustrate the principle behind the arrangement of teeth according to the present invention, and

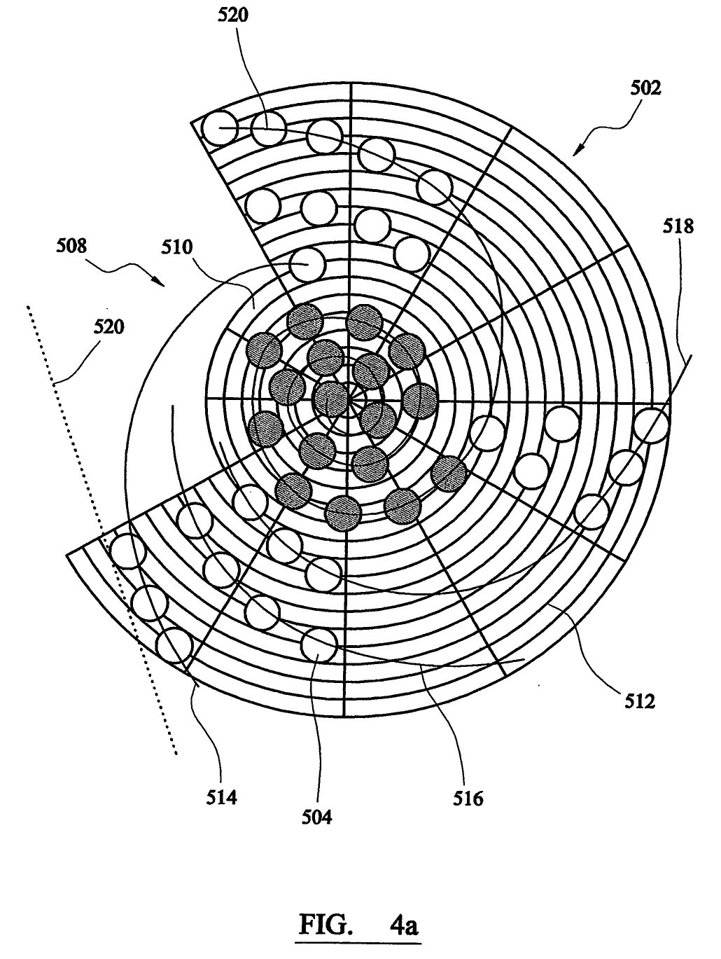

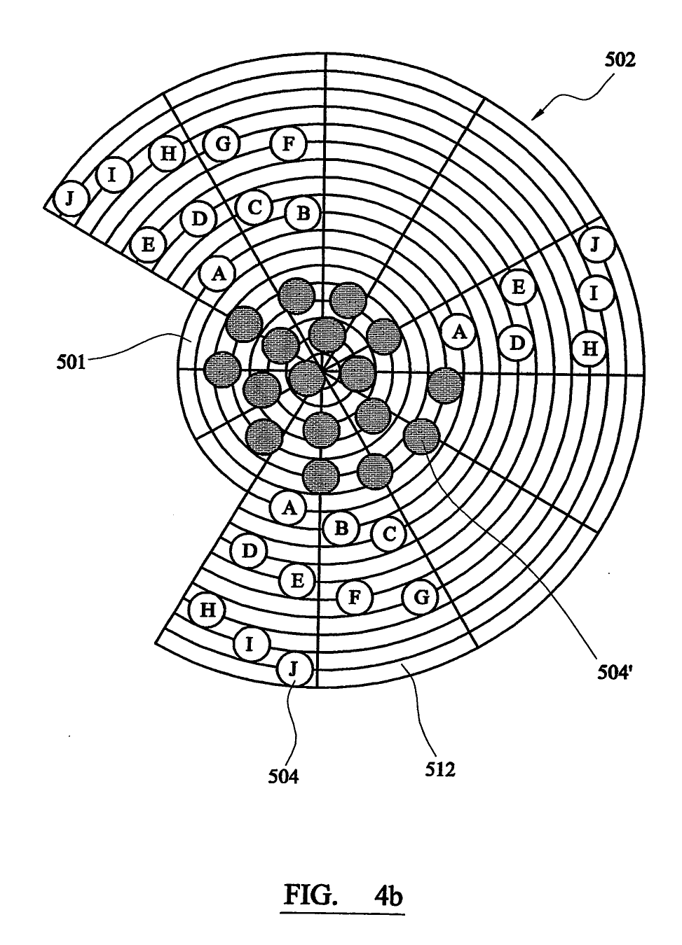

FIGS. 4a and 4b show schematic plan views of a cutting tool according to a second embodiment of the invention.

Referring to the drawings, FIG. 1a shows a schematic plan view of a typical cutting tool 2 having no cut out portions. As can be seen, the teeth 4 of the cutting tool are distributed evenly over the contacting surface of the tool. The general structure of cutting tools is discussed in more detail below in relation cutting tool 102 shown in FIG. 1c.

FIG. 1b shows a “cut down” cutting tool 102 which is equivalent to the cutting tool 2 shown in FIG. 1a apart from it has a portion 108 cut out from it (illustrated by dotted lines). As can be seen, the teeth 104 of the cutting tool 102 are no longer distributed evenly over the contacting surface of the tool.

FIG. 1c shows an isometric view of cutting tool 102. As can be seen, cutting tool 102 comprises a hollow shell formed from an inert metal such as a stainless steel. The material of the shell is about 0.5 mm thick. The shell has cutter teeth 104 which extend outwardly from its external surface, each being formed by cutting perforations into the shell and deforming the material of the shell outwardly.

The shell has a bar 106 extending across it from one side to a diametrically opposite point on the other side, by which the shell can be fastened to a drive instrument which can be used to impart rotation drive to the shell. Clamps by which the bar can be engaged by the instrument are known in this context.

The external surface of the shell forms part of a hemisphere. The angle subtended at the centre of the sphere (of which the surface of the shell forms a part) between the peripheral edge at the opposite ends of the bar 106 is 175°. The shell has one portion 108 cut from it which extends from the peripheral edge of the shell part way towards the pole. The cut out portion is such that the peripheral edge of the shell is intact at the diametrically opposite points where the bar 6 is fastened to the shell.

The external surface of the shell includes an intact region 110 around the pole which is circular when the component is viewed in plan (see FIG. 1b), and whose surface forms part of a sphere. The external surface of the shell further includes an intact portion 112 in which the peripheral edge of the shell is intact.

As discussed above, when cutting tools which have a cut out portion (such as the one shown in FIGS. 1b and 1c) are used, they tend to ‘wobble’ or ‘vibrate’. It has been found that when the net translational force on the tool in the plane which is perpendicular to the axis of rotation, resulting from the accumulated resistance of the teeth when rotated against a rotationally symmetrical cavity in which the tool is a snug fit, is approximately balanced, the vibrating effect experienced is substantially eliminated.

The balancing of the net translation force provided by the teeth is achievable by suitable arrangement of the teeth on the surface of the tool. This is illustrated in FIG. 2 for the tool 102 illustrated in FIGS. 1b and 1c.

FIG. 2 shows a tool 202, which is substantially similar to tool 102 in shape and configuration, having a cutout portion 208, a circular intact portion 210 around the pole, and a further intact portion 212 in which the peripheral edge of the shell is intact. However, the arrangement of teeth 204 on the surface of the tool 202 is such that when the tool is rotated against a bone, the net translational force exerted on the tool by the teeth cutting into the bone, is approximately balanced. As can be seen, the teeth 204 are arranged so that there is an overlap between the teeth as you move from the equator to the pole.

The teeth 204 are arranged on the surface of tool 202 so that each tooth is “balanced” as part of a symmetrical arrangement of 3 or 2 teeth. The teeth 204 shown in FIG. 2 are each labelled with a letter from A-S. Teeth having the same letter are part of the same “symmetry pattern”. For example, there are two teeth labelled “A” which are part of the same “2 symmetry pattern”. This means that they are arranged so that there are two lines of symmetry for those two teeth. There are further teeth arranged in their own 2 symmetry patterns such as teeth “B” to “H”, “J”, “L”, “O”, “P” and “S”. Also, the teeth labelled “I”, “K”, “M”, “N”, “Q”, “R” are each part of their own “3 symmetry patterns”. This means that they are arranged so that there are three lines of symmetry for the three teeth each having the same letter.

FIGS. 3a and 3b illustrate the principle behind the “2 symmetry pattern” and “3 symmetry pattern” strategy of arranging teeth.

FIG. 3a shows a schematic plan view of a cutting tool 302 having no cut out portions and only 2 teeth 304 and 306 arranged in a “2 symmetry pattern”, at a particular moment in time when it is being rotated against a bone, in the direction shown by arrow 308. At that particular moment, the teeth 304 and 306, which are cutting into the bone, are resisting the rotation of the tool, and subsequently forces are exerted on the tool in the directions shown by arrows F₁ and F₂. As the teeth 304 and 306 are arranged in a “2 symmetry pattern”, the net translational force exerted on the tool in the ‘X’ and ‘Y’ planes (i.e. in the plane perpendicular to the axis of rotation) is balanced. This is because force F₁ is equal in magnitude, and opposite in direction, to force F₂.

FIG. 3b shows a schematic plan view of a cutting tool 402 having no cut out portions and only 3 teeth 404, 406, and 408 arranged in a “3 symmetry pattern”, at a particular moment in time when it is being rotated against a bone, in the direction shown by arrow 410. At that particular moment, the teeth 404, 406 and 408, which are cutting into the bone, are resisting the rotation of the tool, and subsequently forces are exerted on the tool in the directions shown by arrows F₃, F₄ and F₅. As will be appreciated, force F₄ can be resolved into X and Y components as shown by arrows F₄ₓ and F_{4y} respectively, force F₅ can be resolved into X and Y components as shown by arrows F₅ₓ and F_{5y} respectively.

As the teeth 404, 406 and 408 are arranged in a “3 symmetry pattern”, the net translation force exerted on the tool in the ‘X’ and ‘Y’ planes (i.e. in the plane perpendicular to the axis of rotation) is balanced. This is because force F3 is equal in magnitude, and opposite in direction, to the net force provided by the X components F₄ₓ and F₅ₓ of forces F₄ and F₅, and also because the Y components F_{4y} and F_{5y} of forces F₄ and F₅ are equal in magnitude and opposite in direction to each other.

Therefore, it can be seen that as shown in FIG. 2, even if all of the teeth are not distributed evenly or symmetrically on the surface of the tool, if all of the teeth are arranged so that they belong to a “2 symmetry pattern” or a “3 symmetry pattern”, then at any point in time, the net translational force exerted on the tool will be balanced and hence the tool is said to be balanced.

Therefore, at any point in time, the “balanced tool” will not move in the direction perpendicular to the axis of rotation of the tool as a result of the force of the bone against the teeth. Hence, as there is no translational movement, no “vibration” will be experienced. This is in contrast to an “unbalanaced tool” which “vibrates” as it is rotated in the bone due to the translational movement of the tool (caused by an unbalance of forces on the tool) in a constantly changing direction.

FIG. 4a shows a further embodiment of a cutting tool 502, which is substantially similar to tool 202 in shape and configuration, having a cutout portion 508, a circular intact portion 510 around the pole, and a further intact portion 512 in which the peripheral edge of the shell is intact. However, in contrast to tool 202, the teeth 504 of tool 502 are arranged in the form of interrupted spirals. The lines 514, 516, 518 and 520 illustrate how the arrangement of the teeth form spirals. Dotted line 520 illustrates the line along which the tool 502 could be cut to allow a further reduction in profile while maintaining a single cut out portion.

FIG. 4b shows how the teeth 504 are arranged on the surface of tool 502 of FIG. 4a so that the net translational force provided on the tool in the plane which is perpendicular to the axis of rotation, resulting from the accumulated resistance of the teeth when rotated against a rotationally symmetrical cavity in which the tool is a snug fit, is approximately balanced.

As can be seen, the black teeth 504′ are not arranged in a “2 symmetry pattern” or “3 symmetry pattern” as described above. Further, the black teeth are not arranged so that the net translational force provided on the tool due to those teeth is approximately balanced. This is because it is difficult to arrange the teeth in the circular intact portion 510 due to the lack of surface area to put the teeth in. However, when the tool 502 is rotated, teeth located near to the axis of rotation will have a slower velocity than those located nearer the equator. Therefore, the force exerted on teeth, by the bone resisting the cutting action of the teeth, will be smaller nearer the axis of rotation than the force exerted on teeth near the equator of the tool.

It has been found that the force on the tool 502 due to teeth 504′ located within the circular intact portion 510 of the tool 502 is negligible in comparison force on the tool due to teeth 504 located outside the circular intact portion. Therefore, it has been found that so long as the teeth 504 outside the circular intact portion 510 are balanced, then even if the teeth 504′ within the circular portion are not balanced, the net translational force is approximately balanced, and therefore the “vibration” experienced is negligible.

1. A cutting tool for preparing a cavity in a bone for receiving a component of an orthopaedic joint prosthesis, in which the shape of the tool is based on a shell having a rotationally symmetrical outer surface, the tool having at least one portion cut out from it, the cut out portion extending from the peripheral edge of the shell toward the pole of the shell, such that the tool has no more than one plane of symmetry passing through the axis of rotation, in which the external surface presents at least two outwardly directed cutting teeth, arranged such that the net translational force on the tool in the plane which is perpendicular to the axis of rotation, resulting from the accumulated resistance of the teeth when rotated against a rotationally symmetrical cavity in which the tool is a snug fit, is approximately balanced.

2. A cutting tool as claimed in claim 1, in which the teeth are arranged into at least two sets of teeth, wherein the teeth of each set are arranged on the contacting surface such that the net translational force on the tool in the plane which is perpendicular to the axis of rotation, resulting from the accumulated resistance of the teeth of each set when rotated against a rotationally symmetrical cavity in which the tool is a snug fit, is approximately balanced.

3. A cutting tool as claimed in claim 2, in which the sets of teeth consist of two teeth.

4. A cutting tool as claimed in claim 2, in which the sets of teeth consist of three teeth.

5. A cutting tool as claimed in claim 2, in which the sets of teeth consist of more than three teeth.

6. A cutting tool as claimed in claim 3, in which the teeth within any of the sets are the same distance from the axis of rotation of the tool, and the length of an are taken between any pair of adjacent teeth within any of the sets of teeth is equal for each pair of adjacent teeth within that set.

7. A cutting tool as claimed in claim 1, in which the teeth are arranged on the contacting surface asymmetrically with respect to the axis of rotation.

8. A cutting tool as claimed in claim 1, in which the teeth are arranged such that their arrangement is that of at least one interrupted spiral.

9. A cutting tool as claimed in claim 1, in which the teeth protrude outwardly from the external surface.

10. A cutting tool as claimed in claim 1, which includes a bar which extends across the interior of the shell, from one side to the opposite other side, by which the shell can be engaged by an instrument for manipulation.

11. cutting tool as claimed in claim 1, in which the shell is intact in a region around the pole.

12. A cutting tool as claimed in claim 11, in which the shape of the external surface of the shell in the region around the pole is generally that of a part of a sphere.

13. A cutting tool as claimed in claim 12, in which the ratio of the distance from the edge of the circular region to the pole to the ratio of the shell to the pole (both distances being measured along the spherical surface of the shell) is not more that about 0.5.

14. A cutting tool as claimed in claim 11, in which teeth located within the region around the pole are not arranged on the contacting surface such that the net translational force on the tool resulting from the accumulated resistance of the teeth when rotated against the bone tissue is approximately balanced.
